(19) United States
(12) Patent Application Publication
(10) Pub. No.: US 2006/0088529 A1
(43) Pub. Date: Apr. 27, 2006

(54) Bispecific monoclonal antibodies to IL-12 and IL-18

(76) Inventors: Stewart Leung, El Cerrito, CA (US); H. Daniel Perez, Kentfield, CA (US); Neil Miyamoto, Corte Madera, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/220,522

(22) Filed: Sep. 8, 2005

Related U.S. Application Data
(63) Continuation of application No. 09/907,968, filed on Jul. 17, 2001, now abandoned.
(63) Continuation of application No. 09/907,960, filed on Jul. 19, 2001, now abandoned.
(60) Provisional application No. 60/219,448, filed on Jul. 20, 2000.

(51) Int. Cl. A61K 39/395; C07K 16/28

(52) U.S. Cl. 424/143.1; 530/388.22

(57) ABSTRACT

A bispecific monoclonal antibody is described which comprises two moieties, one of which comprises an antigen-binding region which is specific for either the IL-12Rβ1 or the IL-12Rβ2 subunit of an IL-12 receptor, and the other of which comprises an antigen-binding region which is specific for either the IL-18R or the AcPL subunit of an IL-18 receptor.

This application is a continuation of U.S. patent application Ser. No. 09/907,960, filed Jul. 19, 2001, which is a U.S. Provisional application having Ser. No. 60/219,448 and was filed Jul. 20, 2000.

### FIELD OF THE INVENTION

This invention relates, e.g., to bispecific antibodies having specificities for the subunits of interleukin-12 and/or interleukin-18 receptors.

### BACKGROUND OF THE INVENTION

Interleukin-12 (IL-12), formerly called cytotoxic lymphocyte maturation factor or NK cell stimulatory factor, and Interleukin-18 (IL-18), formerly called IFN-γ (interferon gamma)-inducing factor, are cytokines which exhibit many biological activities.

The biological activities of IL-12 and IL-18 are mediated by the binding of the cytokines to their cognate receptors on cell surfaces, e.g., T cells, B cells, NK cells or macrophages, in particular on Th precursors. IL-12 receptors comprise at least two subunits, IL12Rβ1 (also known as the beta 1 chain) and IL12Rβ2 (also known as the beta 2 chain). IL-18 receptors comprise at least two subunits: IL-18R (also known as IL-1R-related protein, IL-1Rrp, IL-18Ra, 2FI or the Abinding chain@) and AcPL (also known as accessory protein-like, IL-18-AcPL, IL-18R, or the Asignaling chain@).

### DESCRIPTION OF THE INVENTION

This invention relates, e.g., to a multispecific antibody (e.g., a polyclonal or monoclonal antibody) which is directed against at least one subunit of an IL-12 receptor and/or at least one subunit of an IL-18 receptor. It is to be understood that, although the discussion herein focuses primarily on the IL-12 receptor subunits IL-12Rβ1 and IL-12Rβ2, and the IL-18 receptor subunits IL-18R1 and AcPL, other receptors to which IL-12 or IL-18 bind are also included. The invention encompasses multimeric antibodies which are directed against any combination of the above-mentioned receptors, e.g., against two IL-12 receptor subunits, against two IL-18 receptor subunits, against one IL-12 receptor subunit and one IL-18 receptor subunit, against three or four of the above-mentioned receptor subunits, etc. In a preferred embodiment, the antibody is monoclonal, is bispecific, and is directed against one subunit of an IL-12 receptor and one subunit of an IL-18 receptor (e.g., IL-12Rβ2 and IL-18R1, IL12Rβ2 and AcPL, etc.).

By Amultispecific@ antibody is meant herein an antibody having at least two distinct antibody specificities. Such an antibody can be a single antibody (or an antibody fragment) having multiple specificities, or an aggregate of two or more antibodies (or antibody fragments), each having one or more different specificities. As used herein, when referring to the specific binding of an antibody to an antigen, the terms Abinds to,@ Ahas a binding affinity for,@ Ais specific for,@ and Ais directed against@ are interchangeable and mean that the antibody binds selectively or preferentially to a defined epitope in the antigen (e.g., a polypeptide, polypeptide fragment or peptide).

By Abispecific@ antibody is meant herein a single antibody or antibody fragment having two distinct binding specificities. That is, a bispecific antibody comprises two moieties, each of which comprises a binding region that is specific for a different antigenic target. A Abinding region@ is a portion of an antibody (a polypeptide or a peptide) which comprises an antigen-binding site (a combining site for an antigen).

AAntibodies@ of the invention include polyclonal antibodies, monoclonal antibodies, hybrid or chimeric antibodies, single chain antibodies, fragments such as, e.g., Fab, F(ab=), F(ab)2, or the like. AAntibodies@ can be isolated from any mammalian species, e.g., they can be murine, partially or fully humanized, or human; and they include broadly any immunological binding agent such as, e.g., IgE, IgM, IgA, IgD or, preferably, IgG.

One aspect of the invention is a bispecific monoclonal antibody comprising two moieties, one of which comprises an antigen-binding region that is specific for a subunit of an IL-12 receptor, and the other of which comprises an antigen-binding region that is specific for a subunit of an IL-18 receptor, wherein the two moieties are associated by one or more chemical cross-linkers. An example of such a bispecific antibody is one in which the antigen-binding regions recognize extracellular domains of the receptor subunits and which blocks cytokine binding without stimulating the receptors.

Another aspect of the invention is a bispecific monoclonal antibody comprising two moieties as above, wherein the antigen-binding region of each moiety is appended to a heterologous peptide, and the two moieties are associated via the appended heterologous peptides.

Another aspect of the invention is a bispecific monoclonal antibody comprising an antigen-binding region specific for a subunit of an IL-12 receptor and an antigen-binding region specific for a subunit of an IL-18 receptor, wherein the two regions form (are part of) a single polypeptide chain.

This invention also relates to methods of using the antibody, for instance a method for detecting cells expressing IL-12 and/or IL-18 receptors in a sample which may contain such cells, comprising contacting the sample with a bispecific monoclonal antibody as above which is labeled, and detecting the label.

This invention also relates to a method of treating or preventing a condition (e.g., a pathological condition) associated with expression of IL-12 and/or IL-18, including excessive or inappropriate amounts of those cytokines, and/or with excessive or inappropriate activity of cells possessing IL-12 and/or IL-18 receptors, comprising administering to a patient in need of such treatment an effective amount of a bispecific monoclonal antibody as above.

The multispecific (e.g., bispecific) antibodies of the invention can be prepared in any suitable manner, e.g., 1) by individually preparing antibodies specific for two or more of the receptor subunits, or fragments thereof, and then associating the antibodies, or portions thereof, in various combinations, for example by chemical cross-linking; 2) by preparing individual antibodies as above and then associating them via appended moieties, such as heterologous peptides; 3) by using recombinant methods to prepare a single chain antibody having at least two receptor subunit specificities; or 4) by fusing two or more different cell lines (e.g., hybridomas), each of which produces an antibody directed against one of the receptor subunits, or a fragment thereof, to form a trioma, quadroma or other polydoma, and then isolating multispecific (e.g., bispecific) antibodies which are secreted from the fused cells.

Antibodies specific for a given receptor subunit or a fragment thereof can be obtained according to any suitable method. For example, one can isolate the receptor or fragment, purify it as necessary, and immunize an animal with it. All of these procedures are conventional for a skilled worker.

The IL12Rβ1 and IL12Rβ2 receptor subunits of the IL-12 receptor have been purified, characterized, cloned and sequenced from both mouse and human sources. For procedures to purify, manipulate and/or clone IL12Rβ1 or IL12Rβ2, and/or for a disclosure of their sequences, see, e.g., Chua et al, (1994) J. Immunol. 153, 128; U.S. Pat. No. 5,919,903; Chua et al. (1994) J. Immunol. 153, 128-136; Chua et al. (1995) J. Immunol. 155, 4286-4294; and Presky et al. (1996) Proc. Natl. Acad. Sci. USA 93, 14002-14007. The IL-18R and AcPL receptor subunits of the IL-18 receptor have also been characterized, cloned and sequenced from both murine and human sources, and have been purified from many of them; and they have been at least characterized from other mammalian species such as, e.g., bovine, porcine and various non-human primate sources. For procedures to purify, manipulate and/or clone IL-18R and AcPL, and/or for a disclosure of their sequences, see, e.g., Dinarello (1999). J. Allergy Clin. Immunol. 103, 11-24; Torigoe et al. (1997) J. Biol. Chem. 272, 25, 737-742; Parnet et al. (1996). J. Biol. Chem. 271, 3967-70; EPs 864 585 and 850 952; WO97/31010; U.S. Pat. No. 5,776,731; or Greenfeder et al. (1995) J. Biol. Chem. 270, 13, 757-765; or Born et al. (1998). J. Biol. Chem. 273, 29, 445-450.

Fragments of receptor subunits which can be used as immunogens can be of any size which elicits an antibody response. In a preferred embodiment, fragments corresponding to extracellular domains of the receptors (portions of the receptors which, in an intact cell, are available for binding to a ligand or an antibody) are used. Extracellular domains of IL-12 and IL-18 receptor subunits have been identified and characterized. See, e.g., EP 759466 A2 for the IL-12 receptor subunits, and WO 97/31010 and Born et al. (1998) J. Biol. Chem. 273, 29, 445-50 for the IL-18 receptor subunits. Extracellular domains, fragments thereof, and polypeptides comprising the domains, can be generated from intact receptor subunits by conventional methods, (e.g., with proteases or by chemical cleavage), or can be prepared recombinantly, e.g., as discussed below and in Example 1. Naturally occurring extracellular forms, such as, e.g., Adecoy@ receptors, can also be used.

The receptor subunit polypeptides or fragments thereof can be isolated from any of a variety of sources, e.g., in vivo sources (for example, lung, spleen, epithelial cells, endothelial cells, interstitial cells, chondrocytes, monocytes, granulocytes, lymphocytes, neurocytes, etc.); established cell lines which express one or more of the polypeptides (e.g., hematopoietic cells, including lymphocytes, peripheral blood T cells and NK cells); cells (e.g., lymphoma cells) which secrete one or more of the polypeptides; or recombinant cells which express and, optionally, secrete the polypeptides.

Recombinant cells which express the receptor subunits or fragments thereof can be prepared by conventional methods. As a first step in the generation of such recombinant clones, polynucleotides (e.g., DNA fragments) encoding receptor subunits or fragments thereof can be generated by any of a variety of procedures. For example, they can be cleaved from larger polynucleotides (e.g., genomic sequences, cDNA, or the like) with appropriate restriction enzymes, which can be selected on the basis of published sequences of human and murine IL-18R (see, e.g., Parnet et al., supra and U.S. Pat. No. 5,776,731); human and murine AcPL (see, e.g., Born et al., supra); human and murine IL-12Rβ1 (see, e.g., Chua et al., 1994, 1995 supra); or human and murine IL12Rβ2 (see, e.g., Presky et al., 1996, supra). In another embodiment, polynucleotides encoding receptor subunits, or fragments thereof, can be generated by PCR amplification by selecting appropriate primers based on published sequences such as those above. Methods of PCR amplification, including the selection of primers, conditions for amplification, and cloning of the amplified fragments, are conventional. See, e.g., Innis, M. A. et al., eds. PCR Protocols: a guide to methods and applications, 1990, Academic Press, San Diego, Calif. and Wu et al., eds., Recombinant DNA Methodology, 1989, Academic Press, San Diego, Calif. In another embodiment, polynucleotide fragments encoding receptor subunits, or fragments thereof, can be generated by chemical synthesis. Of course, combinations of the above recombinant or non-recombinant methods, or other conventional methods, can also be employed.

Once a polynucleotide encoding a receptor subunit or a fragment thereof has been isolated, it can be cloned into any of a variety of expression vectors, under the control of a variety of regulatory elements, and expressed in a variety of cell types as hosts, including prokaryotes, yeast, and mammalian, insect or plant cells, or in a transgenic, non-human animal. In a preferred embodiment, the expressed polypeptides are secreted by the cell in order to facilitate purification. Either the natural or a heterologous leader sequence (signal peptide) can be employed to facilitate secretion.

Methods of cloning nucleic acids are routine and conventional in the art. For general references describing methods of molecular biology which are mentioned in this application, e.g., isolating, cloning, modifying, labeling, manipulating, sequencing and otherwise treating or analyzing nucleic acids and/or proteins, see, e.g., Sambrook, J. et al. (1989). Molecular Cloning, a Laboratory Manual. Cold Harbor Laboratory Press, Cold Spring Harbor, N.Y.; Ausubel, F. M. et al. (1995). Current Protocols in Molecular Biology, N.Y., John Wiley & Sons; Davis et al. (1986), Basic Methods in Molecular Biology, Elsevir Sciences Publishing,, Inc., New York; Hames et al. (1985), Nucleic Acid Hybridization, IL Press; Dracopoli, N. C. et al. Current Protocols in Human Genetics, John Wiley & Sons, Inc.; and Coligan, J. E., et al. Current Protocols in Protein Science, John Wiley & Sons, Inc. Other references which, in addition, disclose methods specifically drawn to cloning and characterizing receptor proteins include, e.g., U.S. Pat. Nos. 5,919,903, 5,536,657 and 5,776,731, EP 864 585, and WO 9731010.

Nucleic acids encoding receptor subunits or fragments thereof can also be cloned into plants or animals (e.g., murine species, rabbits, cows, pigs, goats, non-human primates or the like) to generate transgenic species; and the products expressed from the transgenes can be isolated. Methods to make and use transgenic organisms for this purpose are routine and are described, e.g., in Hogan et al., (1986) Manipulating The Mouse Embryo, Cold Spring Harbor Press; Krimpenfort et al., (1991) Bio/Technology 9, 86; Palmiter et al., (1985) Cell 41, 343; Kraemer et al., (1985) Genetic Manipulation of The Early Mammalian Embryo, Cold Spring Harbor Laboratory Press; Hammer et al., (1985) Nature 315, 680; Purcel et al., (1986) Science 244, 1281; Wagner et al., U.S. Pat. No. 5,175,385; and Krimpenfort et al., U.S. Pat. No. 5,175,384.

Preferably, a receptor subunit or fragment thereof of the invention is Aisolated,@ e.g., is in a form other than it occurs in nature, for example in a buffer, in a dry form awaiting reconstitution, as part of a kit or a pharmaceutical composition, etc.

A variety of conventional methods can be used to isolate and/or purify a receptor subunit, or fragment thereof, of the invention. The desired degree of purity may depend on the intended use of the protein. For example, crude preparations of cells transfected with a receptor can be used to generate an antibody, provided that a screening procedure is available which can detect the appropriate monoclonal antibodies. Typically, the protein is substantially purified. The term Asubstantially purified,@ as used herein, refers to a protein which is substantially free of contaminating endogenous materials, such as, e.g., other proteins, lipids, carbohydrates, nucleic acids and other biological materials with which it is naturally associated. For example, a substantially pure molecule can be at least about 60%, by dry weight, preferably about 70%, 80%, 90%, 95% or 99% the molecule of interest. Receptor subunits or fragments thereof can be recovered from cells either as soluble proteins (preferably after having been secreted into the culture fluid) or as inclusion bodies, from which they may be extracted quantitatively, e.g., by 8M guanidium hydrochloride and dialysis. Conventional purification methods which can be used include, e.g., ion exchange chromatography, hydrophobic interaction chromatography, reverse phase chromatography, HPLC, and/or gel filtration. In a preferred embodiment, affinity chromatography is used, e.g., with a column containing IL-12 or IL-18, or another appropriate ligand; an appropriate lectin, such as, e.g., wheat germ agglutinin; or antibodies specific for the IL-12 and/or IL-18 receptors. In a particularly preferred embodiment, a protein is Atagged@ with a moiety, preferably a cleavable one, that can bind to an appropriate affinity column. For example, it can be tagged with poly His (e.g., His₆) to allow rapid purification by met al-chelate chromatography; with a Strep-tag which binds to streptavidin and can be eluted with iminobiotin; with maltose binding protein (MBP), which binds to amylose and can be eluted with maltose; or with any other such moiety which can be separated by affinity chromatography. Alternatively, one can tag one or both of the subunits with epitopes to which antibodies are available, such as the FLAG7 peptide, Asp-Tyr-Lys-Asp-Asp-Asp-Asp-Lys (available from Eastman Kodak Co., Scientific Imaging Systems Division, New Haven, Conn.). Other such antigenic identifiers are described in U.S. Pat. No. 5,011,912 and in Hopp et al. (1988) Bio/Technology 6, 1204. For typical methods of using affinity tags, see, e.g., Recombinant Protein Protocols: Detection and Isolation, Edited by Rocky S. Tuan, Methods in Molecular Biology, Vol. 63, Humana Press, 1997. Combinations of any of the above types of tags can be used, of course.

In a preferred embodiment, individual receptor subunits are expressed separately in recombinant cells. With other methods, in which the receptor subunits are present as mixtures with one or more other receptor subunits, it may be necessary to isolate each receptor subunit individually before introduction into an animal. Such separations can be performed by any of a variety of procedures, e.g., passive elution from preparative, non-denaturing acrylamide gels, or chromatographic techniques, e.g., affinity chromatography of Atagged@ molecules as described above.

The purity of the protein can be determined using standard methods including, e.g., polyacrylaminde gel electrophoresis, column chromatography, and amino-terminal amino acid sequence analysis.

Once receptor subunits or fragments thereof have been isolated, they can be used to immunize animals (e.g., mouse, rabbit, rat, hamster, guinea pig, goat, etc.), thereby generating polyclonal antibodies specific for those proteins. Methods of making polyclonal antibodies well-known to those of skill in the art. See, for example, Green et al. (1992) Production of Polyclonal Antisera, in Immunochemical Protocols (Manson, ed.), pages 1-5 (Humana Press); and Coligan et al. (1992) Production of Polyclonal Antisera in Rabbits, Rats, Mice and Hamsters, in Current Protocols in Immunology, section 2.4.1.

If it is desired to generate monoclonal antibodies, any of a variety of conventional methods can be used. See, for example, Kohler et a. (1975), Nature 256, 495; Coligan et a. (1988), Current Protocols in Immunology, sections 2.5.1-2.6.7; and Harlow et al. (1988), Antibodies: A Laboratory Manual, page 726 (Cold Spring Harbor Pub.). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatograhy, antigen affinity purification and ion-exchange chromatography. See, e.g., Coligan et al., Current Protocols in Immunology, sections 2.7.1-2.7.12 and sections 2.9.1-2.9.3; Barnes et al., (1992), Purification of Immunoglobulin G (IgG), in Methods in Molecular Biology, Vol. 10, pages 79-104 (Humana Press).

Monoclonal antibodies can also be generated recombinantly, using conventional procedures. For example, antibodies of the invention may be derived from antibody fragments isolated from a combinatorial immunoglobulin library. See, for example, Barbas et a. (1991), Methods: A Companion to Methods in Enzymology, Vol. 2, page 119; Winter et al. (1994), Ann. Rev. Immunol. 12, 433, and U.S. Pat. No. 6,004,555.

Methods of in vitro and in vivo multiplication of monoclonal antibodies are well known to those skilled in the art. Multiplication in vitro may be carried out in suitable culture media such as Dulbecco=s Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., osyngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Fragments of either polyclonal or monoclonal antibodies, can be readily generated and isolated and/or purified, using conventional procedures. Antibody fragments can be prepared by proteolytic hydrolysis of the antibody or by expression in a host (e.g., E. coli) of DNA encoding the fragment. Antibody fragments can be obtained by enzyme (e.g., pepsin or papain) digestion of whole antibodies by conventional methods. For example, antibody fragments can be produced by enzymatic cleavage of antibodies with pepsin to provide a 5S fragment denoted F(ab=)₂. This fragment can be further cleaved using a thiol reducing agent, and optionally a blocking group for the sulfhydryl groups resulting from cleavage of disulfide linkages, to produce 3.5S Fab=monovalent fragments and an Fc fragment directly. Such methods are described, for example, by Goldenberg, U.S. Pat. Nos. 4,036,945 and 4,331,647, and references contained therein. See also Nisonhoffetal. (1960), Arch. Biochem. Biophys. 89, 230; Porter (1959), Biochem. J. 73, 119; Edelman et al. (1967), Methods in Enzymology, Vol. 1, page 422 (Academic Press); and Coligan et al., Current Protocols in Immunology, sections 2.8.1-2.8.10 and 2.10.1-2.10.4.

Monoclonal antibodies can be partially or completely humanized, using conventional procedures. For example, a humanized antibody can comprise a variable region of a murine antibody (or just the antigen-binding site thereof) and a constant region derived from a human antibody, or the antigen-binding site of a murine monoclonal antibody and a variable fragment (lacking the antigen-binding site) derived from a human antibody. The use of antibody components derived from humanized monoclonal antibodies obviates potential problems associated with the immunogenicity of murine constant regions.

Humanized monoclonal antibodies can be produced by transferring mouse complementary determining regions from heavy and light variable chains of the mouse immunoglobulin into a human variable domain, and then substituting human residues in the framework regions of the murine counterparts. General techniques for cloning murine immunoglobulin variable domains are described, for example, by Orlandi et al. (1989), Proc. Natl. Acad. Sci. USA 86, 3833. Techniques for producing humanized monoclonal antibodies are described, for example, by Jones et al. (1986), Nature 321, 522; Riechmann et al. (1988), Nature 332, 323; Liu et al. (1987), Proc. Natl. Acad. Sci. USA 84, 3439; Larrick et al. (1989), Bio/Technology 7, 934; Winter et al. (1993), TIPS 14, 139; Jones et al. (1986), Nature 32, 522; Verhoyen et al. (1988), Science 23, 1534; Carter et al. (1992), Proc. Natl. Acad. Sci. USA 89, 4285; and Sandhu (1992), Crit. Rev. Biotech. 12, 437.

In a preferred embodiment, human antibodies are generated by introducing receptors or fragments thereof of the invention into a transgenic mouse in which the immunoglobulin genes have been replaced by large portions of human Ig genes. The antibodies produced are fully human; and the transgenic mice can be used to produce human antibody-secreting hybridomas. Methods of using such transgenic mice are described, e.g., in Green et al. (1994), Nature Genet 7, 13 (1994); Lonberg et al. (1994), Nature 368, 856; and Taylor et al. (1994), Int. Immunol. 6, 579 (1994)

Once antibodies or fragments thereof directed against individual subunits of IL-12 or IL-18 receptors have been isolated, any of a variety of conventional, art-recognized methods can be used to associate (e.g., bind, covalently or non-covalently; couple; attach; cross-link; join; connect; conjugate) two (or more) different antibody moieties to form a bispecific (or multispecific) antibody of the invention. Polyclonal or, preferably, monoclonal antibodies, or fragments thereof, can be used as starting materials. Non-covalent bonds include, e.g., leucine zippers, biotin/avidin interactions, hydrogen bonding, van der Waals forces, hydrophobic interactions, etc. Among possible covalent bonds are, e.g., naturally forming disulfide bonds (e.g., formation of modified Fab or F(ab=)2 fragments), or bonds formed by chemical crosslinking reactions. The attachment can occur in vitro (e.g., in a test tube) or within a cell.

Typical methods of generating, purifying and characterizing bispecific antibodies are disclosed, e.g., in U.S. Pat. Nos. 5,601,819; 6,004,555 and 5,762,930; and Coa et al. (1998), Bioconjugate Chem. 9, 635-644.

As noted above, the general categories of methods which can be used to associate antibody moieties to form the bispecific antibodies of the invention include 1) coupling the moieties by, e.g., chemical crosslinking; 2) appending heterologous peptides to each of the antigen-binding regions to form fusion or hybrid proteins, and joining the fusion or hybrid proteins via the appended peptides; 3) generating single chain antibodies comprising the two antigenic specificities; or 4) somatic fusion of, e.g., hybridomas.

In the first category of methods to generate bispecific antibodies, a variety of types of moieties can be coupled to form bispecific antibodies. For example, two bivalent antibodies, each specific for a different one of the IL-12 or IL-18 receptor subunits, can be separated and the half molecules then rejoined covalently to form a bispecific antibody, using conventional procedures. Such a bispecific antibody comprises a common Fc portion and one Fab portion from each of the parental molecules. Thus, one Fab portion is specific for one of the receptor subunits, and the other is specific for a different receptor subunit. Of course, the starting materials need not be intact, bivalent antibodies. For example, they can be fragments, e.g., Fab fragments, or Fab fragments further comprising one or more heavy chain CH2 and/or CH3 domains (e.g., F(ab=)2 fragments). See FIG. 1A for an illustration of some of the types of bispecific antibodies which can be made by this method. The starting materials can be generated from naturally occurring antibodies or they can be produced recombinantly.

Any of a variety of conventional methods can be used to chemically couple (cross-link) two polypeptide chains (e.g., antibody moieties). Covalent binding can be achieved either by direct condensation of existing side chains (e.g., the formation of disulfide bonds between cysteine residues) or by the incorporation of external bridging molecules. Many bivalent or polyvalent agents are useful in coupling polypeptides. For a description of some methods which can be used to chemically cross-link antibodies, see, e.g., Cao et al. (1988) Bioconjugate Chemistry 9, 635-644; Shalaby et al. (1992) J. Exp. Med. 175, 217-225; Glennie et al. (1987) J. Immunol. 139, 2367-2375; Jung et al. (1991) Eur. J. Immunol. 21, 2431-2435; VanDijk et al. (1989) Int. J. Cancer 44, 738-743; Pierce ImmunoTechnology Catalog & Handbook (1991) E8-E39; Karpovsky et al. (1984) J. Exp. Med. 160, 1686; Liu et al. (1985) Proc. Natl. Acad. Sci. USA 82, 8648; Kranz et al. (1981), PNAS 78, 5807; Perez et al. (1986), J. Exp. Med. 163, 166-178; Brennan (1986) Biotech. 4, 424; and U.S. Pat. Nos. 4,676,980, 6,010,902 and 5,959,083.

In general, the cross-linking agents used are bifunctional agents reactive with E-amino group or thiol groups. These cross-linkers can be classified into two categories: homo- and hetero-bifunctional reagents. Homobifunctional reagents can react, e.g., with free thiols (e.g., generated upon reduction of inter heavy chain or Fab disulfide bonds), and include, e.g., 5,5=-Dithiobis(2-nitrobenzoic acid) (DNTB), and o-phenylenedimaleimide (O-PDM), which can form a thioether bond between two polypeptides having such free thiols. Heterobifunctional reagents can introduce a reactive group onto a polypeptide that will enable it to react with a second polypeptide. For example, N-Succinimidyl-3-(2-pyridyidithio)propionate (SPDP) can react with a primary amino group to introduce a free thiol group. Other chemical cross-linking agents include, e.g., carbodiimides, diisocyanates, diazobenzenes, hexamethylene diamines, dimaleimide, glutaraldehyde, 4-succinimidyl-oxycarbonyl-α-methylα(2-pyridylthio)toluene (SMPT) and N-succinimidyl-S acetyl-thioacetate (SATA).

Spacer arms between the two reactive groups of cross-linkers may have various lengths and chemical compositions. A longer spacer arm allows a better flexibility of the conjugated polypeptides, while some particular components in the bridge (e.g., a benzene group) may lend extra stability to the reactive groups or an increased resistance of the chemical link to the action of various aspects (e.g., disulfide bond resistance to reducing reagents). The use of peptide spacers such as the peptide linkers or linker peptides described below are also contemplated.

In the second category of methods to generate bispecific antibodies, each of two antigen-binding regions, each specific for a different receptor subunit, is appended to another moiety, e.g., any of a variety of heterologous peptides, i.e., peptides which do not occur in immunoglobins (sometimes designated herein as Apeptide linkers@ or Afusion domains@), thereby generating hybrid or fusion proteins. The hybrid or fusion proteins are then associated via the appended moieties. Some of the many types of possible associations via appended moieties are illustrated in FIG. 1B.

In one embodiment, moieties such as biotin and avidin (streptavidin) are complexed to antigen-binding regions, thereby forming hybrid molecules, and, using conventional methods, the hybrid molecules are associated via the biotin and avidin.

In a preferred embodiment, the appended moieties are heterologous peptides (Apeptide linkers@). Among the wide variety of peptide linkers which can be used are the GST (glutathione S-transferase) fusion protein, or a dimerization motif thereof; a PDZ dimerization domain; FK-506 BP (binding protein) or a dimerization motif thereof; a natural or artificial helix-turn-helix dimerization domain of p53; and Protein A or its dimerization domain, domain B. In a most preferred embodiment, the appended peptides are components of a leucine zipper. The leucine zipper moieties are taken from any appropriate source, e.g., the human transcription factors c-jun and c-fos. Of course, such heterologous peptides need not be appended to the ends of antibody molecules. For example, a heterologous peptide can be inserted between two constant domains of a heavy chain.

APeptide linkers@ of the invention encompass variants or fragments of naturally occurring (wild type) peptide linkers (e.g., dimerization domains), provided that the peptide linkers retain the ability to form appropriate associations. Such variants include, e.g., peptides having one or more naturally occurring (e.g., through natural mutation) or non-naturally-occurring (e.g., by deliberate modification, such as by site-directed mutagenesis) modifications, e.g., insertions, deletions and/or substitutions, either conservative or non-conservative.

A peptide linker is preferably long enough to provide an adequate degree of flexibility to prevent the two antibody moieties from interfering with each others=activity, for example by steric hindrance, to allow for proper protein folding and, if necessary, to allow the antibody molecules to interact with two, possibly widely spaced, receptors on the same cell; yet it is preferably short enough to allow the two antibody moieties to remain stable in the cell. Therefore, it may be desirable to modify a peptide linker by altering its length, amino acid composition, and/or conformation, e.g., by appending to it still other Asecondary linker moieties@ or Ahinge moieties.@ Among the many types of secondary linker moieties are, e.g., tracts of small, preferably neutral and either polar or nonpolar, amino acids such as, e.g., glycine, serine, threonine or alanine, at various lengths and combinations; polylysine; or the like. Alternatively, multiples of linkers and/or secondary linker moieties can be used. It is sometimes desirable to use a flexible hinge region, such as, e.g., the hinge region of human IgG, or polyglycine repeats interrupted by serine or threonine at certain intervals.

The length and composition of a peptide linker can readily be selected by one of skill in the art in order to optimize the desired properties of the bispecific antibody, e.g., its ability to bind to a cognate receptor. Conventional assays for binding to IL-12 and/or IL-18 receptors are described, e.g., in Kunikata et al. (1998). Cell. Immunol. 189, 135-143 (IL-18R1); Xu et al. (1998). J. Exp. Med. 188, 1485-1492 (IL-18R1); Rogge et al. (1999). J. Immunol. 162, 3926-3932 (IL-12Rβ2); Gollob et al. (1997). Eur. J. Immunol. 27, 647-652 (IL-12Rβ1); Wu et al. (2000). Eur. J. Immunol. 30, 1364-1374 (IL-12Rβ2); and in Examples 5-7.

Peptide linkers can be appended to antigen-binding regions to form hybrid or fusion proteins by a variety of means which will evident to one of ordinary skill in the art, e.g., chemical coupling as described above (if necessary, following derivatization of appropriate amino acid groups); covalent joining of the peptides by art-recognized methods (e.g., using appropriate enzymes); attachment via biotin/avidin interactions; recombinant methods; or combinations thereof. AHybrid@ proteins of the invention are proteins in which a moiety comprising an antigen-binding region and a moiety comprising a linker peptide are joined via linkages other than peptide linkages (e.g., by chemical coupling or via biotin/avidin interactions). AFusion@ proteins of the invention are proteins in which such moieties are linked by peptide bonds, preferably accomplished by recombinant processes.

Methods of making recombinant fusion proteins are conventional and are described, e.g., in Ashkenazi et al. (1991) PNAS 88, 10535; Byrn et al. (1990) Nature 344, 677; Hollenbaugh et al. (1992) AConstruction of Immunoglobulin Fusion Proteins,@ in Current Protocols in Immunology, Suppl. 4, pp. 10.19.1 to 10.19.11; WO93/10151; and U.S. Pat. No. 5,457,035. Each of the fusion proteins can be expressed independently in a single expression vector, or two or more fusion proteins can be expressed in the same expression vector. Preferably, sequences encoding the two moieties of a fusion protein are in frame.

The antigen-binding regions can be oriented with respect to the appended heterologous peptide so that, when the two antibody moieties are associated, the antigen-binding regions are joined via either their N-termini or their C-termini, provided that the linkage does not interfere with the ability of one or both of the antigen-binding regions to bind to their cognate receptors. In a preferred embodiment, the two antigen-binding regions are joined via their C termini, in order to minimize physical constraints on the Aworking portions@ (the antigen-binding sites) of the molecules, which lie closer to the N-termini. See FIG. 1B for illustrations of some of the possible types of orientations.

Pairs of hybrid or fusion molecules formed as described above can be attached to each other via the appended moieties by non-covalent or covalent bonds. In a preferred embodiment, the attachment occurs intracellularly. Two separate chimeric polynucleotides, each encoding one of two different fusion molecules, are transfected into and co-expressed in the same host cell. Fusion polypeptides so produced are believed to join to one another within the cell or during secretion. They are then purified from a cell lysate or, preferably, are secreted from the cell and are purified from the culture medium. The two fusion proteins can be expressed either from the same expression vector or from two different expression vectors. Generally, fusion proteins are marked with selectable markers, in order to facilitate the selection of transfectants (transformants).

If desired, the relative amounts of two recombinant fusion proteins can be regulated, e.g., by expressing them from promoters of different strengths. For example, if the appended peptide of subunit A forms homodimers at a high frequency, whereas the appended peptide of subunit B forms homodimers at a low frequency, one can drive the formation of the desired heterodimers by expressing much higher levels of subunit B than of A. The optimal relative amounts can be determined empirically by routine experimentation.

The invention also relates to a chimeric polynucleotide encoding a fusion protein as described above, a host cell expressing such a fusion protein, and a method of making such a fusion protein comprising culturing such a cell under conditions in which the fusion protein is expressed and harvesting (recovering) the protein. A fusion protein of the invention can also be made by in vitro translation of a chimeric polynucleotide as above. The invention also relates to antibodies (e.g., monoclonal antibodies) immunoreactive with the novel hybrid or fusion proteins of the invention.

A chimeric polynucleotide of the invention can comprise both coding sequences and regulatory sequences which govern their expression. The nucleic acid sequence corresponding to a moiety of such a fusion protein (e.g., an antigen-binding region or a peptide linker) exhibits substantial identity to the nucleic acid encoding the corresponding wild type molecule, e.g., it comprises a sequence that has at least about 90% sequence identity to the reference sequence, or preferably at least about 95%, or more preferably at least about 98% sequence identity, over a comparison window of at least about 10 to about 100 or more nucleotides. A further indication that two nucleic acids exhibit substantial identity is that the two molecules hybridize to each other under selected high stringent conditions. High stringent conditions are sequence-dependent and will be different with different environmental parameters. Generally, high stringent conditions are selected to be about 5□C. to 20□C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, high stringent conditions will be those in which the salt concentration is at least about 0.2 molar at pH 7 and the temperature is at least about 60□C. Polynucleotides of the invention can include one or more naturally- or non-naturally-occurring modifications, mutations, polymorphisms, etc.; and the nucleic acid can differ from its wild type counterpart with regard to base composition, reflecting the degeneracy of the genetic code.

In the third category of methods to generate bispecific antibodies, recombinant techniques are used to generate a single-chain bispecific antibody. Single-chain antibody binding proteins (sFv) are generated for each of two antigen-binding regions of interest by linking the V_{H} and V_{L} chains, or fragments or variants thereof, with a peptide linker; and the two sets of sFv are then joined, also by a peptide linker, to form a bispecific single chain antibody (bsFv). See FIG. 1C for an illustration of some single chain bispecific antibodies. Recombinant methods in general are described elsewhere herein. Typical methods to generate sFv and are described, e.g., in Whitlow et al. (1991), Methods: A Companion to Methods in Enzymology, Vol. 2, page 97; Bird et al. (1988), Science 242, 423-426; U.S. Pat. No. 4,946,778; Pack et al. (1993), Bio/Technology 11, 1271-77; and Sandhu (1992), Crit. Rev. Biotech. 12, 437. Methods for generating bispecific single chain antibodies, in particular, are described, e.g., in U.S. Pat. No. 5,892,020; Gruber et al. (1994). J. Immunol. 152, 5368-74; Mallender et al. (1994). Biochemistry 33, 10100-10108; Winter et al. (1991). Nature 349, 293-299; Schmidt et al. (1996). International Journal of Cancer 65, 538-546; and Thirion et al. (1996). Eur. J. of Cancer Prevention 5, 507-511.

A variation of single chain bispecific antibodies, diabodies, can also be used. For methods of making such molecules, see, e.g., Holliger et al. (1993), Proc. Natl. Acad. Sci. USA 90, 6444-48 and Cao et al. (1998), Bioconjugate Chem. 9, 635-644.

The two sFv=s in a bispecific single chain antibody can be separated (distanced) from one another by a linker peptide of any length or amino acid composition, most preferably a flexible loop structure, which allows the antibody moieties to lie at an appropriate distance from each other and in a proper alignment for optimal interaction. Typical linker peptides contain tracts of small, preferably neutral and either polar or nonpolar amino acids such as, e.g., glycine, serine, threonine or alanine, at various lengths and combinations; polylysine; or the like. The linker peptide can have at least one amino acid and may have 500 or more amino acids. Preferably, the linker is less than about 100 amino acids, most preferably about 10 to 30 amino acids. Flexible linker domains, such as the hinge region of human IgG, or polyglycine repeats interrupted by serine or threonine at certain intervals, can be used, alone or in combination with other moieties. Routine procedures can be used to select linker peptides and to optimize parameters so that the two antibody moieties are aligned at a distance and in an orientation which allow for optimal functioning. See, e.g., U.S. Pat. Nos. 4,935,233; 4,751,180 and 5,892,020.

The invention also relates to a chimeric polynucleotide which encodes a single chain bispecific antibody molecule as described above; a host cell expressing such a protein; a method of making such a protein, comprising culturing such a cell under conditions in which the protein is expressed and harvesting (recovering) the protein; and an antibody (e.g., a monoclonal antibody) immunoreactive with such a novel single chain polypeptide. A single chain bispecific antibody of the invention can also be made by in vitro translation of such a chimeric polynucleotide.

Properties of such chimeric polynuceotides and variants thereof are as discussed above in relation to polynucleotides corresponding to fusion proteins.

In the fourth category of methods to generate bispecific antibodies, two different clonal cell lines (e.g., hybridomas or lymphocytes) are fused to form a trioma, quadroma or other polydoma, and the bispecific antibodies which are secreted are isolated. Such bispecific antibodies comprise a common Fc portion and one Fab portion from each of the parental cells (e.g., hybridomas). Methods of fusing such cells are conventional and are described, e.g., in U.S. Pat. Nos. 5,959,084, 4,474,893, 5,643,759 and 5,141,736. Typical methods for fusing two established hybridomas to generate a quadroma are described, e.g., in Milstein et a. (1983) Nature 305, 537-540; Stears et al. (1986) Proc. Natl. Acad. Sci. USA 83. 1453-1457; Suresh et al. (1986) Methods Enzymol. 121, 210-228; Suresh et al. (1986) Proc. Natl. Acad. Sci. USA 83. 7989; and U.S. Pat. Nos. 4,474,893 and 5,643,759. Typical methods to fuse one established hybridoma with a lymphocyte from a mouse immunized with a second antigen to generate a trioma are described, e.g., in Nolan et al. (1990) Biochim. Biophys. Acta 1040, 1-11.

Antibody populations produced by such methods contain both homospecific and bispecific molecules. Methods to assay for the presence of bispecific monoclonals are conventional and include, e.g., bridge ELISA assays (see, e.g., Suresh et al. (1986) Proc. Natl. Acad. Sci. USA 83, 7989-93; Koolwijk et a. (1988) Hybridoma 7, 217-225; and De Lau et al. (1989) J. Immunol. 149, 1840-46). Double antigen ELISA may be employed if sufficient quantities of the respective antigens are available. When two different heavy chain isotypes are present in the bsMAb, isotypic specific reagents can be used for detection of hybrid molecules. Furthermore, the supernatants of clones putatively containing bsMAbs can be tested functionally.

In addition to the bispecific antibodies described above, multispecific antibodies can be made by extrapolating any of the above methods, or combinations thereof, to join three or more antibody moieties, in any combination (e.g., antibodies specific for both of the IL-12 receptor subunits plus AcPL; for both of the IL-12 receptor subunits and both of the IL-18 receptor subunits, etc.). Some of the possible variations are summarized in FIG. 2A. In one embodiment, an Fc region may be modified to include a third antigen-binding region. For example part or all of an Fc region may be replaced with a third antigen-binding region. Such modifications can be accomplished with conventional genetic engineering techniques. In other embodiments, bivalent mono- or bi-specific antibodies can be cross-linked to one another in a side-by-side, head-to-head or tail-to-tail orientation. For typical methods to make and use such multimeric antibodies, see, e.g., Tutt et al. (1991) Eur. J. Immunol. 21, 1351-58; Tutt et al. (1991) J. Immunol. 147, 60-69; and Cao et al. (1988) Bioconjugate Chemistry 9, 635-644.

Preferably, a multispecific (e.g., bispecific) monoclonal antibody of the invention is Aisolated,@ as defined above. Methods to isolate and/or purify a bispecific monoclonal antibody of the invention are conventional and are similar to those described above for the purification of receptor subunits or of monoclonal antibodies in general. Among the conventional purification methods which can be used are, e.g., isoelectric focusing, affinity chromatography, including double affinity chromatography (e.g., using sequential mouse anti-idiotype anti-isotype monoclonal antibodies), hydroxylapatite chromatography, ion-exchange chromatography, mimetic affinity methods, gradient thiophilic chromatography, or high performance liquid chromatography. The desired degree of purity may depend on the intended use of the protein.

Bispecific monoclonal antibodies prepared by cell fusion can be obtained from either the supernatant of a hybrid hybridoma (or other polydoma) or from the ascites fluid of a mouse injected with the hybrid hybridoma.

If the method of preparation of a bispecific antibody results in the formation of monospecific as well as bispecific antibodies (e.g., following procedures of chemical coupling), the desired bispecific antibodies can be separated from the monospecific ones by any of a variety of procedures which allow differentiation between the two forms, e.g., passive elution from preparative, non-denaturing acrylamide gels or various conventional chromatographic techniques, e.g., anion-exchange, HPLC, or thiophilic adsorption chromatography (see,e.g., Kreutz et al. (1998). J. Chromatography 714, 161-170). In a most preferred embodiment, each of the antibody moieties is tagged with a different tag, and doubly tagged, bispecific antibodies are separated from singly tagged monospecific antibodies by dual affinity chromatography.

The present invention also relates to methods of using multispecific antibodies of the present invention, e.g., for detection, treatments, research tools, etc.

An antibody of the invention can act either as an antagonist or as an agonist for IL-12 and/or IL-18. These two cytokines, upon binding to a cognate receptor or a subunit thereof, either separately or together, can induce, among others, the following activities: promotion of Tₕ1-type helper cell responses; stimulation of cell proliferation, e.g., of activated T and NK cells; stimulation of the production and/or expression of a number of cytokines, including IFN-γ, e.g., by resting and activated T- and NK-cells; induction of natural killer (NK) cell cytotoxicity; enhancement of cytolytic T-cell responses; inhibition of osteoclast proliferation; tyrosine phosphorylation and activation of Jak2, Tyk2, Stat3, Stat4 or the like; up-regulation of the IL-18 receptor(s) or the IL-12 receptor(s), Fas ligand or ICAM-1; or activation of NF-κB, which can involve activation of, e.g., MyD88, IRAK, TRAF-6, NIK, IKK or IκB.

Antibodies which enhance (e.g., increase, at least to some extent) one or more of the above activities act as Aagonists@ (Aligand-mimicking agents@). Antibodies which inhibit (e.g., decrease, at least to some extent) one or more of these activities act as Aantagonists.@ Of course, an antibody may bind to a cognate receptor, preventing access of a cytokine to the receptor, yet may actually enhance one or more of the above activities; in such a case, the antibody is considered to be an agonist. One of skill in the art can readily determine whether an antibody acts as an agonist or as an antagonist by assaying for any of the above activities, using conventional procedures. See, e.g., Tominga et al. (2000). Intl. Immunol. 12, 151-160; Yoshimoto et al. (1998). J. Immunol. 161, 3400-3407; Xu et al. (1998). J. Exp. Med. 188, 1485-1492; Kunikata et al. (1998). Cell. Immunol. 189, 135-143; Ahn et al. (1997). J. Immunol. 158, 1541-2131; Yoshimoto et al. (1997). Proc. Natl. Acad. Sci. USA 94, 3948-53; Munder et al. (1998). J. Exp. Med. 187, 2103-2108; Otani et al. (1999). Cell. Immunol. 198, 111-119; Hyodo et al. (1999). J. Immunol. 162, 1662-1668; Okamoto et al. (1999). J. Immunol. 3202-3211; Lauwerys et al. (1999). Cytokine 11, 822-830; Bacon et al. (1995). J. Exp. Med. 181, 399-404 (Jak2 and Tyk2); Jacobson et al. (1995). J. Exp. Med. 181, 1755-1762 (Stat3 and Stat4); Kojima et al (1999). J. Immunol. 162, 5063-5069 (NF-κB); Kojima et al. (1998). Biochem. Biophys. Res. Commun. 244, 183-186 (IRAK and Traf-6); Ohtsuki et al. (1997). Anticancer Res. 17, 3253-3258 (Fas ligand); and Kohka et al. (1998). J. Leukocyte Biol. 64, 519-527 (ICAM-1).

While not wishing to be bound to any particular theory of operation of the invention, it is believed that antibodies of the invention can modulate a biological function of a cell bearing IL-12 and/or IL-18 receptor subunit(s) in, for example, one or more of the following ways:

The antibody binds to an extracellular domain of one or more of the IL-12 and/or IL-18 receptor subunits and thereby inhibits the cognate ligand from binding to the receptor.

The antibody inhibits a ligand from inducing one or more of the above-described functions (the antibody acts as an antagonist; the antibody Aneutralizes@ receptor function, or Ablocks@ receptor function).

The antibody stimulates of one or more of the above-described functions (the antibody acts as an agonist).

The antibody up- or down-regulates the expression of one or more of the receptor subunits.

The antibody up- or down-regulates the activities of one or more of the cytokines.

The antibody sensitizes cells bearing IL-12 and/or IL-18 receptor subunits to the effects of cognate cytokines (acts as an agonist).

The antibody inhibits and/or stimulates one or more of the signal transduction functions of a receptor subunit.

The antibody, upon complexing with a receptor subunit, stimulates or inhibits an extracellular activity, e.g., activates serum complement and/or mediates antibody cellular toxicity.

The antibody, if it is associated with a toxin (immunotoxin) or a therapeutic agent (e.g., a drug), delivers the toxin or agent to the surface of the cell, where it then acts at the surface or is taken up by the cell.

This invention relates to a method of treating or preventing a condition (e.g., a pathological condition) associated with expression of IL-12 and/or IL-18, or a receptor or subunit thereof, including excessive or inappropriate amounts of those cytokines, and/or with excessive or inappropriate activity of cells possessing IL-12 and/or IL-18 receptors, comprising administering to a patient in need of such treatment an effective amount of a bispecific monoclonal antibody as above. In a particularly preferred embodiment, the condition is associated with expression of both IL-12 and IL-18, and/or with excessive or inappropriate activity of cells expressing (possessing) both IL-12 and IL-18 receptors.

Activities of IL-12 and/or IL-18, independently or together, include, e.g., the activities noted above. Blocking, enhancing or modifying IL-12 and/or IL-18 activities by contacting their receptors with a bispecific monoclonal antibody of the invention can modulate any of these, or other, activities mediated by IL-12 and/or IL-18, and thus can be used to ameliorate conditions or disorders mediated, directly or indirectly, by these cytokines A disorder is said to be mediated by IL-12 and/or IL-18 when IL-12 and/or IL-18 cause (directly or indirectly) or exacerbate the disorder.

The bispecific monoclonal antibodies of the invention can be used to treat disorders mediated by IL-12 alone, by IL-18 alone, or by both IL-12 and IL-18. Without wishing to be bound by any particular mechanism, in cases in which IL-12 and IL-18 act synergistically (e.g., in certain NK cells, CD4⁺ T cells, B cells or macrophages), the cells may be particularly sensitive (receptive) to treatment with the bispecific monoclonal antibodies of the invention. Furthermore, again not wishing to be bound to any particular theory, it is suggested that, under conditions in which a bispecific monoclonal antibody of the invention binds to IL-12 and IL-18 receptors which are located on the same cell (e.g., simultaneously, sequentially or coordinately), the bispecific antibody exhibits a greater avidity for those cells than does a monospecific antibody. Therefore, under these and other circumstances (e.g., in the presence of other factors), a lower amount of a bifunctional antibody can be required to elicit a given response than that of a monospecific antibody.

Among the many IL-12 and/or IL-18 related conditions which can be treated or prevented by administering to a patient in need thereof a bispecific monoclonal antibody of the invention are a variety of inflammatory conditions (e.g., chronic inflammation), immune disorders (e.g., autoimmune or alloantigen-induced) and allergic diseases. Among the conditions which can be treated or prevented are, e.g. hepatotoxicity associated with endotoxemia, septic shock, autoimmune demyelinating diseases, including multiple sclerosis, rheumatoid arthritis, Crohn=s disease, lupus nephritis, psoriasis, asthma, pernicious anemia, atrophic gastritis, Wegener granulomatosis, discoid lupus erythematosus, ulcerative colitis, inflammatory bowel disease, hyperthyroidism, autoimmune hemolytic anemia, myasthenia gravis, systemic lupus erythematosus, Addison=s disease, Hodgkin=s disease, various leukemias (including, e.g., ALL, CLL, AML and CML), HIV infections, septic shock which results from production or administration of excessive IFN-γ, insulin-resistant and juvenile onset diabetes, atopic dermatitis, and acute or chronic transplant rejection (e.g., Graft-versus-Host disease).

In a preferred embodiment, the bispecific antibodies of the invention are neutralizing antibodies. By Aneutralizing@ is meant herein that binding of an antibody to a receptor subunit inhibits or prevents the binding of a cognate cytokine, and thereby inhibits the activity of the cytokine. A bispecific antibody of the invention may not be neutralizing when it is administered alone, but may become neutralizing when it is co-administered with a second antibody (e.g., an antibody specific for a receptor subunit for which the bispecific antibody is not specific).

In another embodiment, a bispecific antibody of the invention is used to deliver a toxin and/or therapeutic substance (e.g., drug) to a cell which expresses IL-12 and/or IL-18 receptor subunits on its surface. Such an Aagent,@ as a toxin or therapeutic substance can be called, is attached (e.g., conjugated to) the antibody in such a way that it does not substantially disturb the ability of the two antigen-binding regions to bind to their targets. For example, the agent can be attached to an Fc region. Alternatively, when the agent is in the form of a peptide, it can replace all or part of an Fc region, or it can substitute for part or all of an antigen-binding region of a third antibody moiety, forming a structure similar to a third Fab fragment. See FIG. 2B for an illustration of such structures.

An agent of the invention can be any substance which modulates the expression of a cell bearing IL-12 and/or IL-18 receptor subunits on its surface (e.g., provides a therapeutic effect; enhances or suppresses a physiological activity of the cell; or achieves inhibition or suppression of growth, killing, destruction, elimination, control, modification, etc. of the cell). Any effective agent can be used, including an agent which is generally used to treat the conditions noted above. Among the many toxins which can be used are, e.g., ricin (e.g., the A and/or B chain thereof, or the deglycosylated form), poisonous lectins, diphtheria toxin, exotoxin from Psuedomonas aeruginosa, abrin, modeccin, botulina toxin, alpha-amanitan, pokeweed antiviral protein (PAP, including PAPI, PAPII and PAP-S), ribosome inhibiting proteins, especially the ribosome inhibiting proteins of barley, wheat, corn, rye, or gelonin, or ribosome-inactivating glycoprotein (GPIR). Fragments, subunits, muteins, mimetics, variants and/or analogues of such toxins are, of course, known to those of skill in the art and are encompassed by the invention. It is contemplated that all such variants or mutants which retain their toxic properties will be of use in accordance with the present invention. Many possible therapeutic drugs can be used, for example any of a variety of immunosuppressants or immunomodulatory agents, e.g., dexamethasone, cyclosporin or FK506.

Such an agent can be attached to a bispecific antibody of the invention by any of the types of methods described elsewhere herein, e.g., chemical coupling, attachment via biotin/avidin interactions or a peptide linker, recombinant methods, etc.

Of course, antibodies conjugated to such toxic or therapeutic moieties need not be neutralizing (blocking the binding of a cytokine to a receptor). Rather, they can serve to deliver an agent to a target cell, so that the agent can, e.g., exert its effect at the surface of the cell, or be incorporated into the cell.

Antibodies of the invention, whether or not they are associated with toxins or therapeutic agents can, of course, be administered alone or in conjunction with other therapeutic entities.

One of skill in the art can measure activity of the bispecific antibodies of the invention by any of a variety of suitable in vitro or cell culture assays, or in animal models. Several such assays are discussed herein. Further in vivo methods include, e.g., systems for evaluating graft vs. host reactions (see, e.g., Fanslow et al. (1990) Science 248, 739-741 and animal models (e.g., the EAE model)for autoimmune demyelinating diseases such as, e.g., multiple sclerosis. For a description of animal models of MS, see, e.g., Gold et al. (2000). Mol. Med. Today 6, 88-91 and Swanborg (1995). Clin. Immunol. Immunopathol. 77, 4-13. For a description of some methods of using the EAE animal model, see, e.g., Leonard et al. (1995). J. Exp. Med. 181, 381-386 and Wildbaum et al. (1998). J. Immunol. 161, 6368-6374.) See also Dinarello (1999) J. Allergy Clin. Immunol. 103, 11-24.

Bispecific antibodies of the invention can be administered using conventional doses and delivery methods, such as those described for other, comparable therapeutic agents.

Dosages to be administered can be determined by conventional procedures known to those of skill in the art. See, e.g., The Pharmacological Basis of Therapeutics, Goodman and Gilman, eds., Macmillan Publishing Co., New York. In general, effective dosages are those which are large enough to produce the desired effect, e.g., blocking the binding of endogenous IL-12 and/or IL-18 to the natural receptor, or delivery of a toxin or drug. The dosage should not be so large as to cause adverse side effects, such as unwanted cross-reactions, anaphylactic reactions, and the like. Factors to be considered include the activity of the specific antibody/agent involved, its metabolic stability and length of action, mode and time of administration, drug combination, rate of excretion, the species being treated, and the age, body weight, general health, sex, diet, and severity of the particular disease-states of the host undergoing therapy. For example, appropriate therapeutic regimens for a bispecific antibody of the invention involve administration to a patient of a dose of between about 0.1 mg/kg and about 10 mg/kg.

Appropriate methods of administration include parenteral and non-parenteral routes of administration. Parenteral routes include, e.g., intravenous, intraarterial, intraportal, intramuscular, subcutaneous, intraperitoneal; intraspinal, intrathecal, intracerebroventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection.

Ingredients, including excipients, diluents and/or carriers, for pharmaceutical compositions useful for the various modes of administration are conventional in the art, and are described, e.g., in Remington=s Pharmaceutical Sciences, 18th ed., Mack Publishing Company (1990). The bispecific antibodies can be formulated, e.g., in a pharmacologially acceptable liquid, solid or semi-solid carrier, linked to a carrier or targeting molecule (e.g., antibody, hormone, growth factor, etc.) and/or incorporated into liposomes, microcapsules or controlled release preparations (including cells which express the heterodimeric receptors) prior to administration in vivo.

The invention also relates to methods of detecting a cell which expresses an IL-12 and/or IL-18 receptor subunit, and/or of detecting the receptor subunits, themselves, comprising contacting a sample which may contain such a cell (or receptor subunit) with a bispecific monoclonal antibody of the invention, which is labeled (i.e., comprises a detectable moiety). Typically, in cells which express receptor subunits, extracellular domains of the receptor subunits are present at the surface of the cells and are available for binding to the antibody. Conventional methods can be used to label and detect the antibodies. Typical labels include, e.g., radioisotopes, radionuclides, phosphorescent or fluorescent entities, bioluminescent markers, stains, or the like. Such assays can be quantitative, of course. Although not wishing to be bound by any theory, it is suggested that bispecific antibodies of the invention exhibit a particularly high avidity for cells bearing both target receptors, and thus specifically label such cells in preference to cells expressing only one of the receptors.

In one embodiment of the invention, assays are used to determine whether an agent of interest causes an increase or decrease in the amount of IL-12 and/or IL-18 receptor subunits on the surface of a cell (e.g., human or murine cells; in a test tube, in culture, or in an animal), and/or whether it modulates (inhibits or enhances) the biological activity of a receptor subunit (e.g., its ability to bind to an antibody). Alternatively, an assay can indirectly monitor the amount of IL-2 and/or IL-18 in a cell, by monitoring the amount of free receptor which is available for binding to a cytokine or antibody (i.e., to determine the level of receptors which have not been saturated by the cytokines). Assays of the invention can be used, e.g., for experimental characterization of an agent; for screening for potentially therapeutic agents; for the diagnosis of diseases which can be indicated by the levels of IL-18 in bodily fluids (e.g., radiodiagnosis); or to monitor the effects of treatment.

### BRIEF DESCRIPTION OF THE DRAWINGS

FIG. 1(A-D) shows some examples of bispecific antibodies. Panel A illustrates chemical cross-linking; Panel B illustrates linkage via appended moieties; Panel C illustrates single chain polypeptides; and Panel D illustrates molecules formed by cell fusion.

FIG. 2(A-B) shows some examples of multispecific antibodies. Panel A antibodies having three or more specificities; Panel B illustrates multispecific antibodies comprising toxic or therapeutic peptides.

FIG. 3 shows the effect of α-IL-12 treatment in EAE.

FIG. 4 shows the effect of α-IL-18 treatment in EAE.

FIG. 5 shows the effect of α-IL-12 plus α-IL-18 treatment in EAE.

FIG. 6 shows that IL-2 and IL-18 can synergistically induce IFN-γ production in CD14-depleted human peripheral blood mononuclear cells (PMBC).

FIG. 7 shows that IL-2 and IL-18 can synergistically induce IFN-γ production in CD3⁺ and CD4⁺ T cells.

FIG. 8 shows IFN-γ production in IL-18 stimulated KG-1 cells and IL-12 stimulated NK-92 cells.

FIG. 9 shows an in vivo model to test the activity of α-IL-18 monoclonal antibodies.

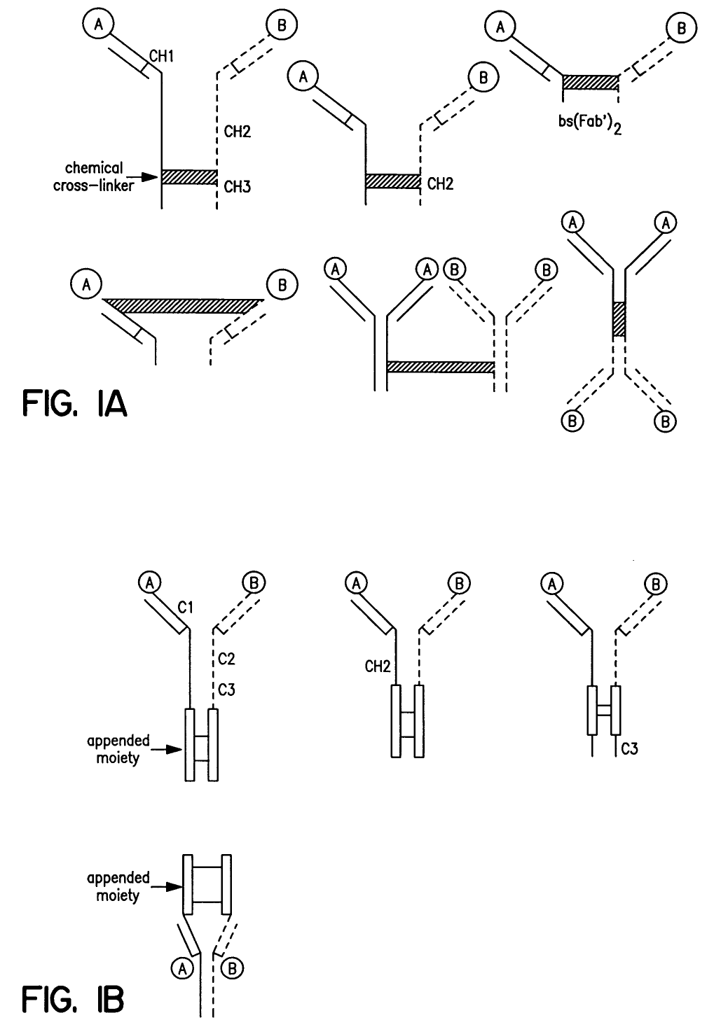

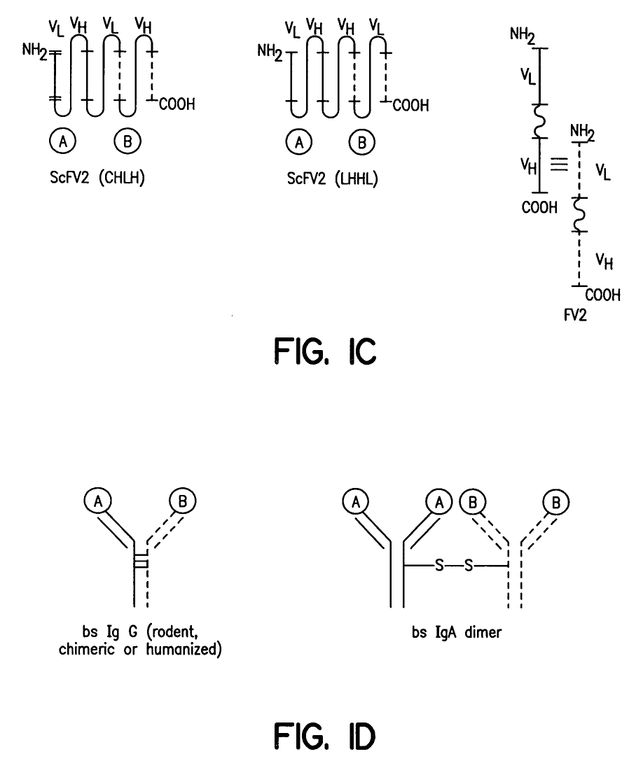

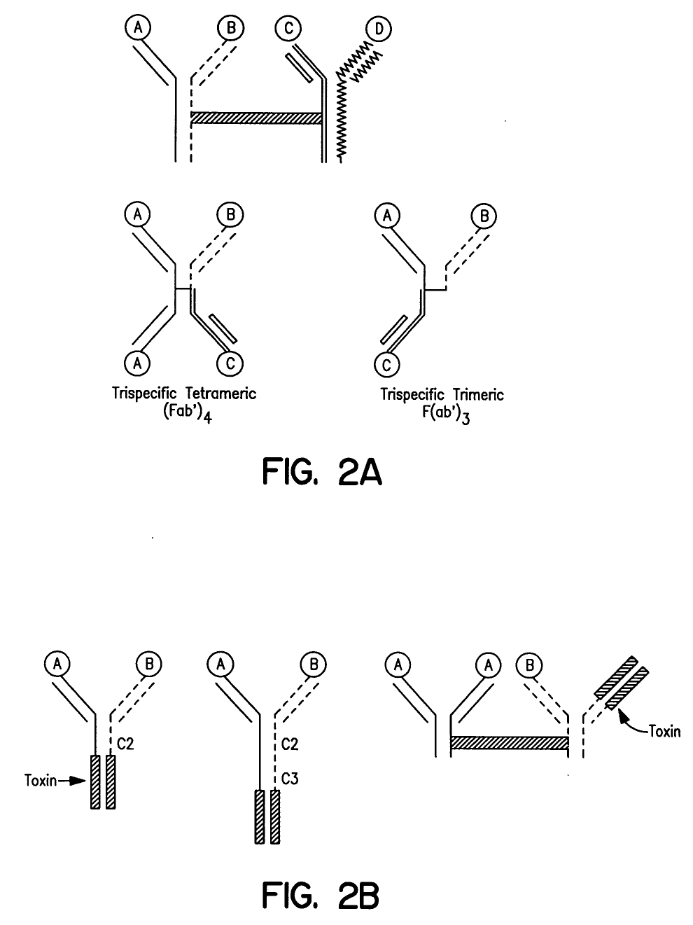

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosure of all applications, patents and publications, cited above or below and in the figures are hereby incorporated by reference.

### EXAMPLES

1. Cloning of Full-length Human IL-12 and IL-18 Receptor Subunits

Human IL-12 and IL-18 receptor subunits (e.g., full length molecules) are cloned following standard procedures by reverse transcription-polymerase chain reaction (RT-PCR) using gene-specific primers and total RNA isolated from conA-activated, IL-12 and IL-18 stimulated, CD14-depleted PBMC. RT is performed using the Clontech AAdvantage RT-for-PCR kite using an oligo-dT primer, and subsequent PCR is performed using primers corresponding to the 5= and 3= ends of the coding sequences. The full-length cDNA is cloned, via restriction enzymes sites engineered into the primers, into the eukaryotic expression vectors pcDNA3.1(−)MYCHISB or pcDNA3.1(−)PUR (Invitrogen). For example, full-length human IL-12Rβ2 and IL-18R cDNAs are cloned.

Human IL-12Rβ1: The open reading frame of the full-length human IL-12Rβ1 cDNA (Accession # U03187) is from position 65 to 2053, encoding a protein of 662 AA, with a signal peptide from AA 1 to 24.

Human IL-12Rβ2: The open reading frame of the full-length human IL12Rβ2 cDNA (Accession # U64198) is from position 641 to 3229, encoding a protein of 862 AA, with a signal peptide from AA 1 to 27.

Human IL-18R: The open reading frame of the full-length human IL-18R cDNA (Accession # U43672) is from position 25 to 1650, encoding a protein of 541 AA, with a signal peptide from AA 1 to 19.

Human AcPL: The open reading frame of the full-length human ACPL cDNA (Accession # AF077346) is from position 484 to 2283, encoding a protein of 599 AA with a signal peptide from AA 1 to 14.

2. Cloning of Extracellular Domains of Human IL-12 and IL-18 Receptor Subunits

The extracellular domains of human IL-12 and IL-18 receptor subunits are cloned as described in Example 1, except that the 3= primer corresponds to the C-terminus of the extracellular domain of the respective proteins. For example, the extracellular domains of the human IL-12Rβ2 and IL-18R cDNAs are cloned.

Human IL-12Rβ1: The extracellular domain of human IL-12Rβ1 is from AA 1 to 540.

Human IL-12Rβ2: The extracellular domain of human IL-12Rβ2 is from AA 1 to 622.

Human IL-18R: The extracellular domain of human IL-18R is from AA 1 to 329.

Human AcPL: The extracellular domain of human AcPL is from AA 1 to 356.

3. Generation of Monoclonal Antibodies to Human IL-12 or IL-18 Receptor Subunits

Methods to generate antibodies (e.g., neutralizing antibodies) to cell surface receptor molecules are well documented in the field of immunology, and are described, e.g., in Methods in Molecular Biology, Vol. 45, Monoclonal Antibody Protocols, ed. by Davis, W. C., Human Press, Inc., 1995, and Current Protocols in Immunology, ed. by Coligan, J. E. et al., J. Wiley & Sons, 1992). One method of immunization is to inject mice with an antigen that is a purified extracellular domain of a receptor, which is expressed in bacteria, insect cells, yeast, or mammalian cells. A preferred method of immunization is to express the recombinant full-length human receptor(s) in a mouse pre-B cell line and then inject the stably transfected mouse cells as antigen into the same (or a closely related) strain of mice from which the line was derived. This method has been described for IL-12Rβ2 (Gollub, J. A. et al., Eur. J. Immunol. 27:647-652, 1997). In other situations, cells from other species may be used, e.g., as described for the IL-12 receptor using PHA-activated human PBMC (Gately et al., U.S. Pat. No. 5,853,721, Dec. 29, 1998). Immunized mice are boosted and bled according to standard protocols.

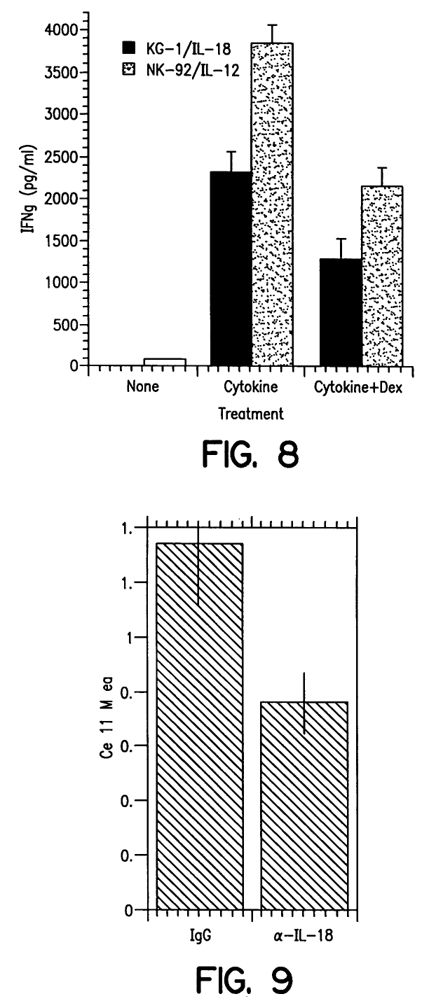

Mice are immunized with a mouse pre-B cell line stably transfected with both human IL12Rβ1 and IL12Rβ2 (IL-12 receptor) or both human IL-18R and AcPL (IL-18 receptor). The expression of functional receptors on the stably transfected cells is verified by binding studies with radio-labeled ligands. The sera from immunized mice are screened for the presence of antibodies to the target receptor (either IL-12 or IL-18) using the same cell line used for the immunization. The non-transfected cells are used as negative control. One can screen for neutralizing activity of the antibodies by inhibition of ligand binding to the cells expressing the receptor. This can be the same cell line used for the immunization, or another cell line expressing the target receptor. For the IL-12 and IL-18 receptors, one can use the human natural killer line NK-92 and the human myelomonocytic line KG-1, respectively (see Example 7 and FIG. 8). In cells expressing a functional receptor, the sera can also be tested for their ability to inhibit IFN-γ production.

Spleen cells from mice producing antibodies to the target receptor are fused with a mouse myeloma cell line to generate antibody-secreting hybridomas. To facilitate the generation of bi-specific monoclonal antibodies (see Example 8 below), different myeloma lines with different drug resistant phenotypes can be used for fusion with the α-IL-12 and α-IL-18 receptor antibody producing cells. The culture media from the hybridomas are screened for antibodies to the target receptor as described above. The hybridomas that produce neutralizing antibodies are cloned, and antibody preparations are generated from either large-scale hybridoma culture media or ascites fluid from mice injected with the hybridomas. Antibodies are purified by affinity column chromatography, using either Protein A/G or specific antigen bound to the column support matrix.

To generate human monoclonal antibodies, transgenic mice carrying portions of the human immunoglobulin heavy chain and kappa light chain loci and lacking their endogenous counterparts are used for the initial immunization. Medarex=s HuMAb-Mouse technology, which was originally developed by GenPharm Int., Inc., has been successfully used to generate high affinity human antibodies.

4. Effect of α-IL-12 Alone, α-IL-18 Alone, and α-IL-12 Plus α-IL-18 in EAE

To assess the effects of IL-12 and IL-18 in rodent models of multiple sclerosis (MS), we investigated the effects of antibodies to IL-12 and IL-18 in PLP-induced, adoptive transfer EAE in SJL mice. The α-IL-12 and α-IL-18 antibodies used were commercially available and demonstrated to be neutralizing in vitro, due of their ability to inhibit IFN-γ production in the murine Th1 clone Ae7 in response to IL-12 and IL-18, respectively. The α-IL-18 antibody was also shown to be neutralizing in vivo, due to its ability to reduce liver damage in P. acnes/LPS-treated mice (see Example 10 and FIG. 9).

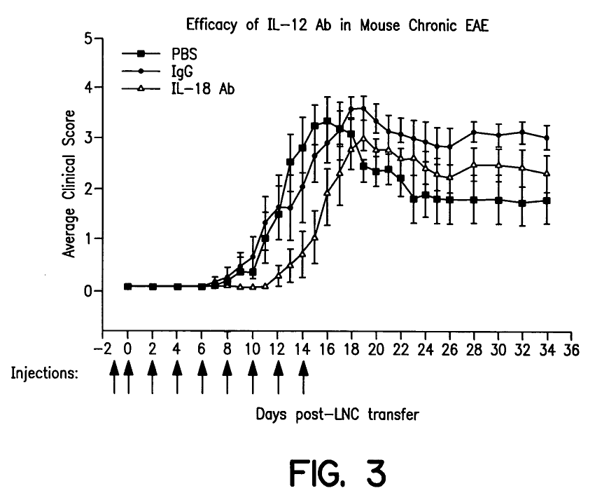

α-IL-12 alone: FIG. 3 shows that α-IL-12 treatment delays the onset and reduces the disease scores as compared to PBS or control IgG. See also experiments reported by Leonard et al., J. Exp. Med. 181: 381-386, 1995, using a different antibody.

A commercially available, goat α-mouse IL-12 polyclonal antibody was injected i.p at 200 μg/mouse on the days indicated. There was a delayed onset of disease and a reduced peak clinical score in the α-IL-12 treated mice as compared to mice receiving PBS or treated with an equal amount of goat IgG. The clinical score of the α-IL-12 treated mice was significantly different (p<0.05) from the IgG treated mice (n=9-10).

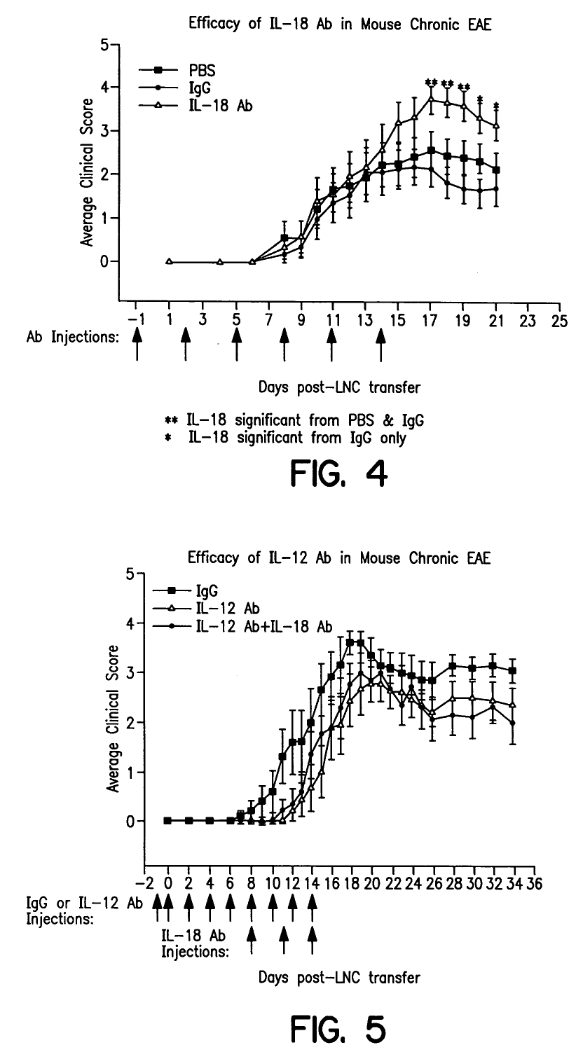

α-IL-18 alone: FIG. 4 shows that α-IL-18 treatment has no effect on disease onset, but significantly exacerbates disease scores as compared to PBS or control IgG2a.

A PLP-induced, adoptive transfer EAE study was performed in SJL mice with a commercially available, rat α-mouse IL-18 polyclonal antibody. On the indicated days, 250 μg/mouse was injected i.p. There was no difference in the onset of disease in the α-IL-18 treated mice as compared to control mice receiving PBS or treated with an equal amount of IgG. However, the average clinical score for the α-IL-18 treated mice was significantly higher than either the PBS or IgG control mice.

α-IL-12 plus α-IL-18: FIG. 5 shows that combined α-IL-12 and α-IL-18 treatment has the same protective effect as α-IL-12 treatment alone.

A commercially available, goat α-mouse IL-12 polyclonal antibody was injected i.p, at 200 μg/mouse, as indicated. Another group of mice received α-IL-12 plus a commercially available, rat α-mouse IL-18 monoclonal antibody. On the indicated days, 250 μg/mouse was injected i.p. There were delayed onsets of disease and reduced peak clinical scores in the α-IL-12 and α-IL-12 plus α-IL-18 treated mice as compared to mice treated with an equal amount of goat IgG. The α-IL-12 and α-IL-12 plus α-IL-18 treated mice were significantly different (p<0.05) from the IgG treated mice (n=9-10), but not from each other.

5. Synergistic Induction of IFN-γ Production by IL-12 and IL-18 in Human CD14-depleted PBMC

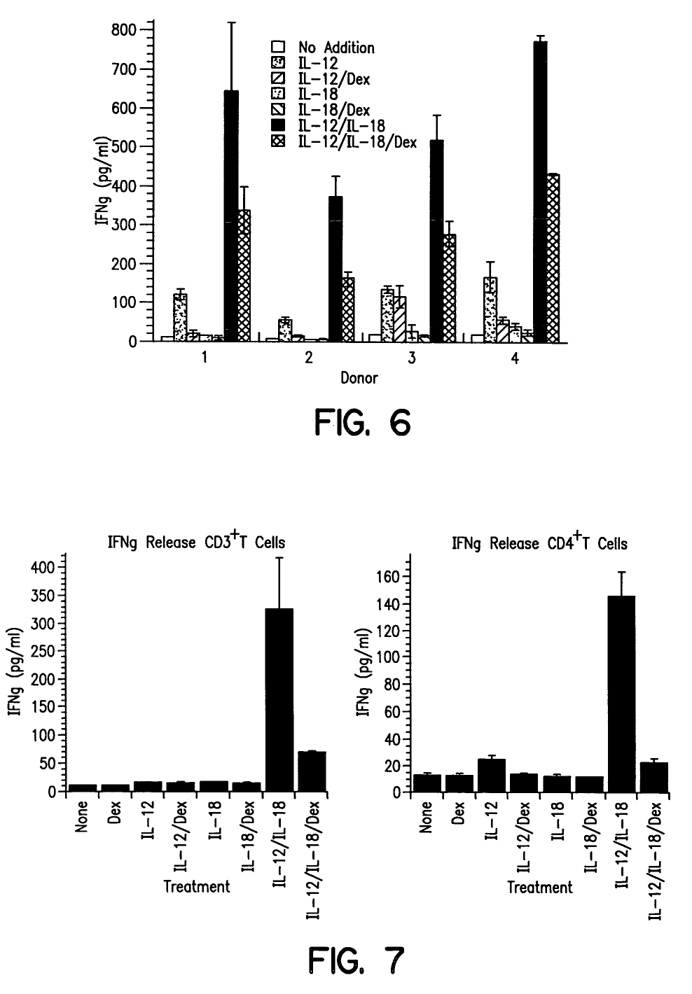

The cytokines IL-12 and IL-18 can synergize for production of pro-inflammatory Th1 effector cytokines, such as IFN-γ. FIG. 6 shows an assay that shows synergistic induction of IFN-γ production in conA-primed, IL-12/IL-18 stimulated CD14-depleted human PBMC.

Purified, conA-primed human CD14-depleted PBMC from four normal donors were plated at 2.5×10⁵ cells/ml in 96-well plates. As indicated, DEX (20 nM), IL-12 (10 pM), or IL-18 (50 nM) were added. The cells were incubated for 16 to 24 hours, and the amount of IFN-γ in the culture medium was determined using the Biosource Cytoscreen IFN-γ ELISA kit.

6. Synergistic Induction of IFN-γ Production by IL-12 and IL-18 in Human CD3+ and CD4+ T Cells

CD3+ and CD4+ T cells were purified from conA-primed CD14-depleted human PBMC, and their ability to respond to IL-12 and IL-18 was investigated. As shown in FIG. 7, both CD3+ and CD4+ T cells produce IFN-γ in response to IL-12 and IL-18 together, but not to either cytokine alone.

Purified, conA-primed human CD3+ or CD4+ T cells (>95% purity) were plated at 5×10⁵ cells/ml in 96-well plates. As indicated, 50 μl of DEX (20 nM), IL-12 (10 pM), or IL-18 (50 nM) were added. The T cells were incubated for 16 to 24 hours, and the amount of IFN-γ in the culture medium was determined using the Biosource Cytoscreen IFN-γ ELISA kit.

7. Induction of IFN-γ Production by IL-12 in NK-92 Cells and by IL-18 in KG-1 Cells

Two assays show the bioactivity of IL-12 and IL-18 alone. NK-92 is a natural killer line derived from a patient with malignant Hodgkin=s lymphoma. KG-1 is a myelomonocytic line derived from a patient with acute myelogenous leukemia. NK-92 and KG-1 cells constitutively express the IL-12 and IL-18 receptors, respectively. As shown in FIG. 8, IL-12 and IL-18 induce IFN-γ production in NK-92 cells and KG-1 cells, respectively.

KG-1 cells were cultured in serum-free medium for 24 hours, and then plated at 1×10⁶ cells/ml in 96-well plates. As indicated, IL-18 (50 nM) or DEX (20 nM) were added. NK-92 cells were cultured in the presence of 10 U/ml IL-2, and plated at 2.5×10⁴ viable (Trypan Blue negative) cells/ml in 96-well plates. As indicated, IL-12 (10 pM) or DEX (20 nM) were added. The KG-1 and NK-92 cells were incubated for 16 to 24 hours, and the amount of IFN-γ in the culture medium was determined using the Biosource Cytoscreen IFN-γ ELISA kit.

8. Method to Generate Bi-specific Monoclonal Antibodies

Bi-specific monoclonal antibodies are generated by a variety of methods known to those skilled in the art of making monoclonal antibodies (see, e.g., Cao et al., M. R. Bioconjugate Chem. 9: 635-644, 1998). A preferred method to generate a bi-specific human monclonal antibody that recognizes, binds, and inhibits both the IL-12 and IL-18 receptor is to generate a quadroma, which is formed by fusing two hybridomas (described in Cao, Y. et al. J. Immunol. Methods 187:1-7, 1995). The bi-specific antibody can be purified from the quadroma by gradient thiophilic affinity chromatography (as described, e.g., in Kreuntz, F. T. et al. J. Chromatog. 714:161-170, 1998).

9. In Vitro Assays to Demonstrate Activity of Bi-specific Monoclonal Antibodies

In vitro assays that can be used to demonstrate the neutralizing activity of either monoclonal antibodies or bi-specific human monoclonal antibodies to the IL-12/IL-18 receptors are described, e.g., in Examples 5, 6, and 7 and FIGS. 6, 7 and 8.

10. In Vivo Models to Demonstrate Activity of Bi-specific Monoclonal Antibodies

In vivo models that can be used to demonstrate the neutralizing activity of either monoclonal antibodies or bi-specific human monoclonal antibodies to the IL-12/IL-18 receptors include, e.g., LPS-induced endotoxic shock in Balb/C mice; P. acnes/LPS-induced liver damage in nude mice (see FIG. 9); PLP-induced adoptive transfer EAE in SJL mice (see FIGS. 3-5); and type II collagen-indced arthritis in DBA/1 mice.

FIG. 9 shows, for example, that mice treated with RDI α-IL-18 display lower pathology in the P.acnes/LPS liver damage model. Male nu/nu Balb/C mice were injected with 1 mg heat killed P.acnes (iv); RDI α-IL-18 or normal rat IgG was injected 7 days later; LPS (1 μg) was injected 1 h after the antibodies; mice were sacrificed 24 h later; and livers were subjected to histological analysis. The livers from animals treated with RDI α-IL-18 antibody had lower pathological scores. These changes were statistically significant (Significance Level: 5%, n=6 per group, P-value=0.0346).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modification of the invention to adapt it to various usages and conditions.

1. A bispecific monoclonal antibody comprising two moieties, one of which comprises an antigen-binding region which is specific for either the IL-12Rβ1 or the IL-12Rβ2 subunit of an IL-12 receptor, and the other of which comprises an antigen-binding region which is specific for either the IL-18R or the AcPL subunit of an IL-18 receptor.

2. A bispecific monoclonal antibody of claim 1, wherein one of the antigen-binding regions is specific for IL-18R1 and the other is specific for IL-12Rβ2.

3. A bispecific monoclonal antibody of claim 1, wherein one of the antigen-binding regions is specific for AcPL and the other is specific for IL-12Rβ2.

4. A bispecific monoclonal antibody of claim 1, wherein each of the antigen-binding regions is appended to a heterologous peptide, thereby forming a hybrid or fusion protein, and wherein the hybrid or fusion proteins are associated via said appended peptides.

5. The bispecific monoclonal antibody of claim 4, wherein each appended peptide is part of a leucine zipper.

6. The bispecific monoclonal antibody of claim 1, wherein the two moieties are associated by chemical cross-linking.

7. The bispecific monoclonal antibody of claim 1, wherein the two moieties form a single polypeptide chain.

8. The bispecific monoclonal antibody of claim 6, wherein the chemical cross-linking is between two Fc molecules.

9. The bispecific monoclonal antibody of claim 1, wherein the IL-12 and IL-18 receptors are human.

10. (canceled)

11. A method of making a bispecific monoclonal antibody of claim 1, comprising associating the two moieties by cross-linking them chemically, by joining them via an appended heterologous peptide linker, by forming a single linear polypeptide chain with recombinant methods, or by fusing two hybridoma cells, each of which expresses one of said moieties.

12. A chimeric polynucleotide which encodes a fusion protein comprising an antigen-binding region that is specific for an IL-12 receptor subunit appended to a heterologous peptide, and/or a fusion protein comprising an antigen-binding region that is specific for an IL-18 receptor subunit appended to a heterologous peptide.

13. An expression vector comprising a chimeric polynucleotide of claim 12.

14.-15. (canceled)

16. A polynucleotide which encodes a bispecific antibody of claim 7.

17.-19. (canceled)

20. A method of making a bispecific monoclonal antibody of claim 1, comprising fusing two hybridoma cells, each of which expresses one of the two moieties.

21. A method of making a bispecific monoclonal antibody of claim 1, comprising immunizing-a transgenic mouse that can produce human antibodies with a polypeptide which comprises an extracellular domain of a subunit of an IL-12 receptor or with a polypeptide which comprises an extracellular domain of a subunit of an IL-18 receptor.

22. A method for inhibiting the effects of IL-12 and/or IL-18, comprising administering a bispecific monoclonal antibody of claim 1 to a mammal.

23. A method of treating a pathological condition associated with expression of IL-12 and/or IL-18, or with excessive or inappropriate activity of cells possessing IL-12 and/or IL-18 receptors, comprising administering to a patient in need of such treatment an effective amount of a bispecific monoclonal antibody of claim 1.

24. The method of claim 23, wherein the patient is human.

25. The method of claim 23, wherein the pathological condition is an autoimmune dysfunction or an inflammatory condition or rheumatoid arthritis or multiple sclerosis.

26. (canceled)

27. A method for suppressing IL-12 and/or IL-18 mediated inflammation or an IL-12 and/or IL-18 mediated immune response in a mammal, comprising administering to a patient in need of such treatment an effective amount of a bispecific monoclonal antibody of claim 1.

28. The method of claim 27, wherein the mammal is human.

29. A method for delivering a toxin or therapeutic agent to a patient in need of such treatment, comprising administering to the patient an effective amount of a bispecific monoclonal antibody of claim 1 which further comprises a toxin or therapeutic agent.

30. A method of detecting a cell expressing an IL-12 and/or IL-18 receptor subunit, comprising contacting a sample which may contain such a cell with a bispecific monoclonal antibody of claim 1, which is labeled.
